(19) United States
(12) Patent Application Publication
(10) Pub. No.: US 2003/0009088 A1
(43) Pub. Date: Jan. 9, 2003

(54) Monitoring system for patients

(76) Inventors: Uwe Korth, Stolberg (DE); Alexander Wolf, Aachen (DE)

Correspondence Address: HOWSON AND HOWSON, ONE SPRING HOUSE CORPORATION CENTER, BOX 457, 321 NORRISTOWN ROAD, SPRING HOUSE, PA, 19477, US

(21) Appl. No.: 10/115,884

(22) Filed: Apr. 4, 2002

(30) Foreign Application Priority Data
Apr. 4, 2001 (DE) DE 201 05 926.6
Nov. 17, 2001 (DE) DE 101 56 167.9

(51) Int. Cl. A61B 5/00

(52) U.S. Cl. 600/300; 128/903; 128/904

(57) ABSTRACT

The invention concerns a monitoring system for patients with at least one measuring device for medical and diagnostic values of the patient such as blood pressure, pulse, body temperature, blood sugar and the like, which measuring device is carried and operated by the patient. According to the invention it is proposed that the measuring device feature at least one transmitter for the wireless transmission of the recorded values to at least one local receiver. In this arrangement, it is particularly advantageous if the receiver is integrated into a mobile phone, which sends the recorded values to a central data processing device via a mobile telephone network.

### DESCRIPTION

The invention concerns a monitoring system for patients with at least one device for the recording of medical and diagnostic values of the patient such as blood pressure, pulse, body temperature, blood sugar, body weight and the like, or for the recording of a patient activity, which device is carried or operated by the patient and generates a set of data as a result of the recorded values or activity. Depending on the measurements to be taken and on the activities to be recorded, the set of data may contain a multitude of different measuring values, a multitude of consecutive measuring values, one measuring value or even only one signal.

Portable or mobile measuring devices for patients are known, which, after having been manually started or at preset intervals, record certain measuring values of the human or animal body. These serve in particular for the long-term monitoring of patients with hypertension and hypotension. The measured values are stored and possibly also evaluated in a device-internal memory. For an analysis it is required to read this memory. For this purpose an interface is required in order to connect the device to a home PC or a computer of the treating physician. This can be rather complicated and requires a measure of skill and expert knowledge on the part of the patient and/or physician.

For effective treatment of a disease or for the long-term treatment of a condition that can only be alleviated it is required, in addition to monitoring the condition, that the prescribed drugs be taken in a timely and regular fashion. While devices are known that, through acoustic signals, remind one to take the drugs, this does not constitute a real control. The invention concerns such a device, as well. However, the invention concerns in particular a measuring device for the recording of medical and/or diagnostic data, so that in the following the term ‘measuring device’ will be used frequently, which shall not be construe

The invention is based on the task of designing a monitoring system of the initially mentioned type in such a manner that the data transfer from the device to an evaluating unit is facilitated. In particular, the patient should not be involved in it.

The task is solved according to the invention in that the measuring device or apparatus features at least one transmitter for the purpose of transmitting the sets of data with the recorded values in a wireless fashion to at least one receiver. The receiver is connected with a unit, which evaluates or initially just registers and stores the measured values, for example a local computer. The data transfer can thus happen entirely independent from the patient's location, position and condition. Of course, the measuring device may feature an intermediate memory so that the data transfer happens only after the values have been recorded completely and properly.

It is useful for the data recording system to contain a memory and/or a computer and/or an output unit in order to be able to store and/or process and/or evaluate the received sets of data and/or to issue them to a medium, in particular a data carrier. In this manner, a statement can be made and an evaluation of the measured values can be performed in situ. This is useful e.g. in hospitals.

For the purpose of covering a larger area, several receivers and/or data recording devices can exist. The receivers may be connected to the data recording device through wiring or in a wireless manner. It is also possible that several receivers could be connected to one data recording device.

According to a more advanced embodiment form of the invention, it is provisioned that the data recording device be connected with a central data processing device for evaluation and further analysis. This central data processing device may be positioned in a location that is different from the location of the data recording device. In this manner it is possible for the physician or the monitoring institution to continuously monitor the data of one or several patients.

It is advantageous for the data recording device to be connected with the data processing device via a telecommunication link, in particular a radio connection of an existing mobile phone network or via a server. In this arrangement, the data recording device comprises a dialing device for the purpose of dialing into the data processing device and of transmitting the sets of data of the patient via this telecommunication line. Already installed telecommunication connections permit such data transfer without any problems.

It can be provisioned that the data transfer from the data recording device to the data processing device be performed immediately after a set of data has been received and/or at preset time intervals and/or at least one preset point in time. In this fashion, the costs of the data transfer can be kept low.

On principle, it suffices if the measuring device and the data recording device are connected with each other for signal transmission from the measuring device to the data recording device in a one-directional manner. However, it can also be provisioned that the measuring device and the data recording device feature a wireless bidirectional signal connection. In this arrangement it is useful if the data recording device comprises at least one time signal emitter, which, at preset time intervals, transmits a starting signal to the measuring device to perform the measurements. The measuring device can also contain a timer signal emitter in order to send, at preset time intervals, a signal and/or the measured values to the data recording device. On principle, however, the measured values can be recorded continuously, in a quasi-continuous manner or at regular intervals.

According to another embodiment of the invention, the measuring device operates directly in conjunction with a mobile phone. In this arrangement it is provisioned that the measuring device transmit the set of data of measured values to the mobile phone by means of existing wireless transmission interfaces such as infrared, radio or by way of BlueTooth® technology. The mobile phone is equipped or modified in such a way that subsequently a long distance data transfer e.g. via SMS (Short Message Service) or similar is performed directly to the data processing device via a mobile phone network. Such a modification or programming of the mobile phone can, e.g., be accomplished through the SIM-card or multi-media connection, the so-called MMC bay. Interference with the device electronics of the mobile phone is not absolutely necessary.

The algorithms necessary for the evaluation are set up in the data processing device and the measured values can be evaluated. Depending on the embodiment, the data is processed for long distance data transfer in the measuring device or in the mobile phone. The patient only needs to start the measuring process or only to make sure that the measuring device is positioned within receiving range of the corresponding mobile phone. It is also possible for the measuring device itself to be equipped with a mobile phone transmitter, which transmits the recorded set of data directly to the data processing device.

According to an additional embodiment of the invention, it is provisioned that a comparing device inside the data recording device and/or inside the data processing device compare the recorded measuring values of the patient with standard values and that an alarm signal exist that emits an alarm signal or a transmits a corresponding message, if the measured value deviates from a presettable tolerance value. Such a message can, e.g., also be sent through an SMS to a mobile phone of a doctor or caretaker. Such functions are possible within a mobile phone network. In this manner, a patient's critical condition can be quickly diagnosed over long distances. Emergency measures can then be initiated quickly and effectively.

Another advantage must be seen in the fact that in combination with a mobile phone the patient remains entirely mobile. Measuring values can also be sent to the data processing device while traveling and be evaluated there. Above all, it is possible during so-called long-term monitoring over a period of 24 hours or over a longer period of time to make the measured values available to the treating physician continuously or e.g. just in time for the next visit of the patient.

Below, the invention is explained by means of a schematic drawing. The figures show:

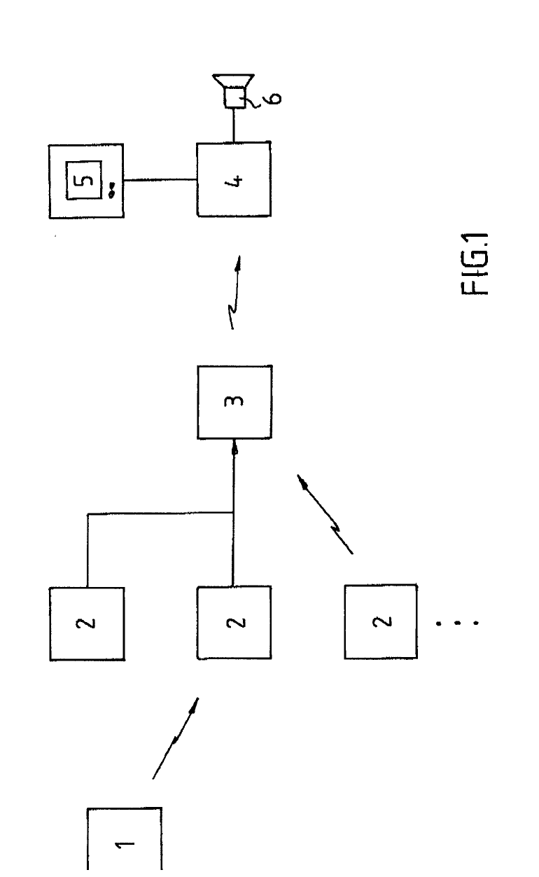

FIG. 1, a schematic block diagram of a monitoring system according to the invention; and

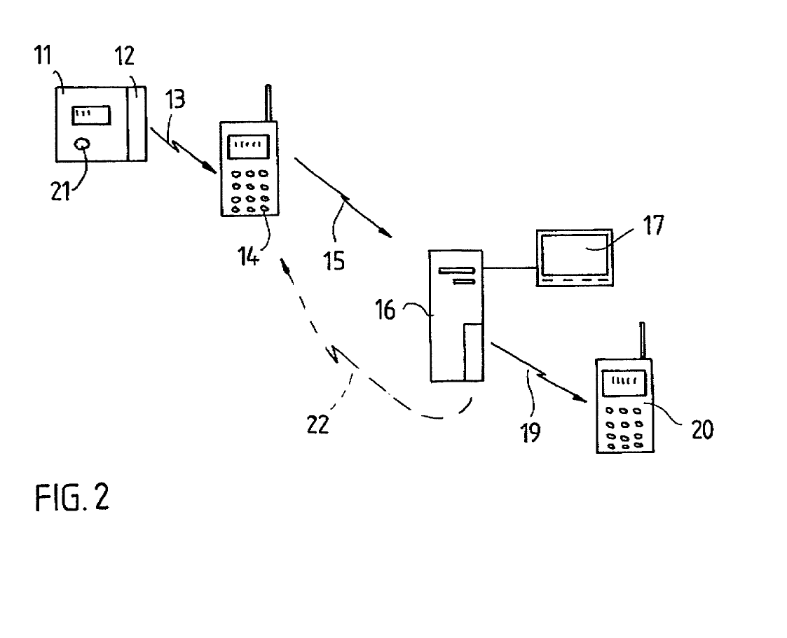

FIG. 2, a schematic block diagram of an alternate embodiment of the invention.

In the embodiment according to FIG. 1, at least one measuring device 1 is provisioned, which the patient carries and which in time intervals, after manual actuation or in certain time intervals or at certain points in time, records measuring values such as, e.g., blood sugar content, blood pressure, body temperature and pulse. These values can be stored intermediately. Measuring device 1 is connected via a wireless signal transmission with at least one receiver 2. In large areas or in different rooms several receivers 2 may exist, each of which receives the signal of the measuring device. A receiver may also be positioned in the patient's vehicle. Of course, several patients can be monitored simultaneously with the system. The only requirement for this is that the transmitted signals must be coded or different frequencies must be provided.

Receivers 2 are wire-connected or wireless connected with a data recording device 3. If a receiver is installed in the patient's vehicle, a wireless signal connection to the data recording device can e.g. be established via the mobile telephone that is usually installed in the vehicle. The receivers can be connected with the data recording device by means of an intermediate device or by means of a multiplexer that coordinate the various receivers and their readiness to receive. Accordingly, the measured values can be transmitted to data recording device 3 wirelessly, either in certain time intervals, after performance of a measurement, or continuously. There, the measured values can be processed, modified, evaluated or stored.

In addition, data recording device 3 is connected with a data processing device 4 via a telecommunication means e.g. a modem or telephone or mobile phone. The data processing device can be located at the place of the physician or in a hospital. A display system 5 exists that displays the transmitted data. A signal device 6 may exist that issues an alarm signal when it identifies dangerous or risky measured values. This makes real-time monitoring of one or several patients possible.

In the monitoring system according to FIG. 2, measuring device 11 features a transmitter 12 that can be put into signal connection 13 with a mobile phone 14. For this purpose, the interfaces that already exist on a mobile phone, such as infrared or radio interfaces, can be used. It is also possible to equip the mobile phone with an appropriate adapter that communicates with measuring device 11.

Measuring device 11 builds up this signal connection 13 after the measured values are present. However, it can also be provisioned for these and possibly other measuring values for a certain period of time, e.g., for a day or a week, to be stored in a memory and forwarded to the mobile phone at a presettable time. It is also useful for a control circuit or a control function to exist, which, e.g., controls the complete transfer of the measured values to the mobile phone. In this event, it is useful if the measured values remain stored in the memory at least for as long as it takes to transmit them completely.

Mobile phone 14 is equipped with an automatic dialing function in such a manner that, after having completely received the measuring values to be transferred, a connection 15 is established to a central data processing device 16. There, the measured values are evaluated and, e.g., output on a monitor 17, or a printer or an electronic data carrier. It is also possible for the sets of data containing the measured values to be initially stored intermediately within the mobile phone until they are accessed and transferred. Access can also be initiated by the data processing device.

Data processing device 16 is capable of performing a comparison or evaluation, in order to check the received measured values for critical values. If critical measuring values or significant deviations are found, an optical alarm signal can be displayed on monitor 17. It can also be provisioned for the data processing device to contain a telecommunication device 18 through which a further signal connection 19 is established for example to a mobile phone 20 of the treating physician. The selected measuring value can then be displayed on the display of mobile phone 20 so that critical values can be checked in a nearly real time fashion. This makes fast therapeutic measures possible.

Measuring device 11 features a central operating button 21, by means of which measurements can be started and performed. Subsequently, signal connection 13 is established automatically, in order to transmit the measured values to the mobile phone. From there the further transmission is performed also automatically, e.g. to the display device of the treating physician. In this manner, the operation of the monitoring device is substantially facilitated. It is, of course, also possible for the measuring device to initiate the measurements by itself at preset points in time or at preset time intervals. The patient does not have to take any active measures.

The above-described measuring device can be embodied as a wrist device or as a mobile measuring device with a sleeve to hold it on the arm or as a stationary device. It can also be a component of a scale for measuring body weight. When measuring blood sugar, the measuring device can contain a corresponding measuring device for the patient's blood. To the extent that activities need to be monitored, which the patient must perform regularly or at certain points in time, the measuring device can be integrated into a corresponding device. For example, the pill dispenser for the daily ration of drugs can be equipped with a signal transmitter via which a corresponding message that a drug has been taken out of the dispenser is sent via the mobile phone to the physician. Actual monitoring is possible. In this context in particular but also when recording and/or transmitting sets of data it is useful if the set of data in question is stamped with the actual time or coded accordingly. For this purpose, a real-time emitter can be provisioned in the device.

In addition, it is possible that the performance of the measurements and/or the action and in particular the taking of the medication can be monitored in a nearly real-time manner. It is possible to provision for the data processing device to monitor measuring values and/or messages within a time frame or in a time-specific manner and generate an alarm message also in the event of missing or insufficient measuring values or messages. This message can be sent to the monitor 17 and directly to mobile phone 20 of the treating physician or back to mobile phone 14 of the patient via telecommunication device 18. This signal transmission 22 is shown in a dashed line in FIG. 2. Accordingly, the patient is constantly reminded of the performance of the measurement or actions such as taking the medication or similar. This additional function can be installed without any further measures together with the monitoring of the patient that is required anyway.

1. Monitoring system for patients with at least one device (1, 11) for the recording of medical and diagnostic values of patients such as blood pressure, pulse, body temperature, blood sugar, body weight and similar or for the recording of a patient's activity, which device is carried or operated by the patient and generates, based on the values or the activity recorded, a set of data, wherein the device features at least one transmitter for the purpose of transmitting the set of data in a wireless manner to at least one local receiver (2).

2. Monitoring system according to claim 1, wherein the receiver is integrated into a mobile phone (14), which transmits the set of data, via a mobile phone network, to a central data processing device (16).

3. Monitoring system according to claim 1 or 2, wherein several receivers (2) exist in order to cover a larger spatial area.

4. Monitoring system according to one of claims 1-3, wherein the receiver or receivers (2) are connected with a local data processing device (3).

5. Monitoring system for patients with at least one device (11) for the recording of medical and diagnostic patient data, such as blood pressure, pulse, body temperature, blood sugar, body weight and the like, or for the recording of an activity of a patient, which device is carried or operated by the patient and which generates, based on the recorded values or activity, a set of data, wherein the device features at least one transmitter, which is embodied as a mobile phone transmitter, for the purpose of transmitting the set of data via a mobile telephone network to a data processing device.

6. Monitoring system according to one of claims 1-5, wherein local data recording device (3) and/or data processing device (4, 16) includes a memory and/or computer and/or an output unit for the purpose of storing and/or processing and/or evaluating and/or outputting the received sets of data to a medium, in particular a data carrier.

7. Monitoring system according to one of claims 1-6, wherein the data recording device is connected with a data processing device via a telecommunication connection, in particular a radio connection of an existing mobile phone network or via a server.

8. Monitoring system according to claim 7, wherein the data are transferred from local data recording device (3) and/or mobile telephone (14) and/or mobile telephone transmitter to the data processing device immediately upon receiving a set of data and/or in presettable time intervals and/or at least at a certain point in time.

9. Monitoring system according to one of claims 1-8, wherein at least one timer signal emitter is provisioned, which emits, in preset time intervals or at predetermined points in time, a start signal to the device to start performing the measurement(s).

10. Monitoring system according to one of claims 1-9, wherein the timer signal emitter is integrated into the device.

11. Monitoring system according to one of claims 1-10, wherein the device contains a real-time clock in order to stamp each set of data with the actual time of its generation.

12. Monitoring system according to one of claims 1-11, wherein a comparison device in the data recording device and/or in the data processing device compares the recorded measured patient values with standard values, and wherein an alarm signal emitter (6) is contained, which issues an alarm message when values deviate from a preset tolerance value.

13. Monitoring system according to one of claims 1-12, wherein the device or the transmitter comprises a controlling device that controls the transmission of the recorded values to the receiver.

14. Monitoring system according to one of claims 1-13, wherein the device contains a memory for at least the last recorded set of data and in particular for several consecutive sets of data of the recorded values, in which memory at least the last set of data remains stored until it has been transmitted to the receiver.

15. Monitoring system according to one of claims 2-14, wherein the mobile phone and/or the local data recording device are equipped with an auto-dialing function, in order to transmit the set of data to the data processing device automatically.

16. Monitoring system according to one of claims 1-15, wherein the device features a single operating button, after the one-time activation of which the measurement(s) and the subsequent data transmission to the data recording or data processing device are started and performed.

17. Monitoring system according to one of claims 1-16, wherein the device is integrated into a medication dispenser and generates a set of data with a corresponding message, when the dispenser is operated.

18. Monitoring system according to one of claims 1-17, wherein the data recording device and/or data processing device features at least one control circuit, which records the actual and/or timely receipt of a set of data with values and/or messages and which generates an alarm message, if a message is not received or incorrectly transmitted.

19. Monitoring system according to one of claims 2-18, wherein the alarm message is transmitted to the patient's mobile phone and or to a further communication installation.
